(19) United States
(12) Patent Application Publication
(10) Pub. No.: US 2024/0306936 A1
(43) Pub. Date: Sep. 19, 2024

(54) Wearable Collector for Noninvasive Sampling of Exhaled Breath Biomarkers, Pathogens or Viral Loads

(71) Applicant: THE BOARD OF TRUSTEES OF THE LELAND STANFORD JUNIOR UNIVERSITY OFFICE OF THE GENERAL COUNSEL,, Stanford, CA (US)

(72) Inventors: Fernando Soto, Millbrae, CA (US); Utkan Demirci, Stanford, CA (US); Mehmet Ozgun Ozen, Sunnyvale, CA (US)

(21) Appl. No.: 18/275,677

(22) PCT Filed: Apr. 13, 2022
(86) PCT No.: PCT/US2022/024681

§ 371 Date: Aug. 3, 2023

Related U.S. Application Data
(60) Provisional application No. 63/181,049, filed on Apr. 28, 2021.

(51) Int. Cl. A61B 5/097; A61B 5/00; A61B 5/08; A61B 10/00

(52) U.S. Cl. CPC A61B 5/097; A61B 5/082; A61B 5/6803; A61B 2010/0087; A61B 2562/12

(57) ABSTRACT

Airborne transmission of exhaled virus can rapidly spread by increasing disease progression from local incidents to pandemics. Due to the COVID-19 pandemic, states and local governments have enforced the use of protective masks in common and work areas to minimize disease spread. Here, the function of protective face coverings is leveraged towards COVID-19 diagnosis. A user-friendly, affordable, and wearable adhesive collector was developed. This non-invasive platform is integrated into protective masks towards collecting airborne virus exhaled from individuals' breath over the wearing period. After sampling or collecting, the enriched pathogen can be extracted from the collector for further analytical evaluation. To validate this design, qualitative colorimetric loop-mediated isothermal amplification (LAMP), quantitative reverse transcription polymerase chain reaction (RT-PCR), and antibody-based dot blot assays were performed to detect the presence of SARS-CoV-2. This platform enables easy sampling of current SARS-CoV-2 and other airborne diseases of future pandemics.

### FIELD OF THE INVENTION

This invention relates to wearable collector devices and methods for noninvasive sampling of exhaled breath biomarkers, pathogens or viral loads.

### BACKGROUND OF THE INVENTION

Diagnosis of the severe acute respiratory syndrome coronavirus 2 (SARS-CoV-2) responsible for the coronavirus pandemic (COVID-19) remains time-consuming and a challenge for clinicians and researchers. Most of the patients are asymptomatic until advanced stages when individuals experience severe symptoms. However, in the early stages, when infected patients are asymptomatic, the virus spreads through breath to other individuals without their awareness.

Without the capacity to accurately quantify who is infected, governments have established large-scale quarantine measures that have had catastrophic effects on the global economy. Constant screening in the general population and healthcare professionals would enable early detection of asymptomatic patients.

The current gold standard method to determine if a patient is infected with SARS-CoV-2 relies on amplifying the virus' genetic material using quantitative reverse transcription polymerase chain reaction (RT-PCR). Other approaches include the use of lateral flow immunoassays (LFIs) based on anti-coronavirus antibodies (IgG and/or IgM) or CRISPR technology to detect SARS-CoV-2. Nevertheless, the antibody-based methods cannot detect the current status of infection, while CRISPR based technology is not implemented for routine diagnosis. Recent works have reported the development of portable sensors for saliva or nasopharyngeal swab.

Despite their great utility, one of the main bottlenecks when screening patients is the type and method of sampling. Current sampling methodologies include collecting nasopharyngeal swabs, blood or saliva, in sterile containers. These sampling methods can cause patient discomfort (nasal swabs and needles) and, in some cases, requires extra processing steps for isolating the few viruses present in it (saliva). On the other hand, aerosol or filter samplers rely on long-term exposure for sampling, leading to time-dependent virus collection and enrichment compared to one-time sample collection as happens with nasopharyngeal, blood or saliva. Such sampling devices have been crucial to understanding virus generation due to their high sensitivity and isolation capabilities. However, most of these collectors are bulky, have long sampling time requirements, and require trained professionals to perform sampling.

The present invention provides technology to address at least some of these shortcomings and concerns.

### SUMMARY OF THE INVENTION

The present invention provides a wearable breath-based noninvasive sampler patch allows collecting large quantities of virus/pathogens over prolonged periods of time combining the resilience and commodity of a disposable test. In one example, the wearable breath-based noninvasive sampler integrates a detachable (medical) adhesive and a porous membrane for target enrichment in the sample. This wearable breath-based sampler is attached on the inner surface of face-covering and collects large quantities of viruses over prolonged periods of time. The flexible device is compatible with any mask or protective equipment, can sustain constant mechanical deformation, and can be made in large quantities. After sampling, the enriched pathogen was extracted from the collector for further gold standard analytical evaluation. (RT-PCR, LAMP).

In one embodiment, the surface can be integrated with various sensors to detect the targets quantitatively and/or qualitatively on the surface itself and outcomes can be noticed via visual indicators detectable to the naked eye or via quantitative tools externally.

Embodiments of the invention have advantages over existing devices and methods in that they are stand-alone collector devices for personal use, non-invasive when compared to nasal swabs, low-cost and make scalable fabrication possible adaptable to any type of protective mask.

### BRIEF DESCRIPTION OF THE DRAWINGS

FIG. 1 shows according to an exemplary embodiment of the invention a flexible substrate of the wearable collector device.

FIG. 2 shows according to an exemplary embodiment of the invention a flexible substrate of the wearable collector device where the mask attachment section has a different shape compared to the one in FIG. 1.

FIG. 3 shows according to an exemplary embodiment of the invention a side view of the flexible substrate as shown in FIGS. 1-2 with the addition of a double-sided adhesive.

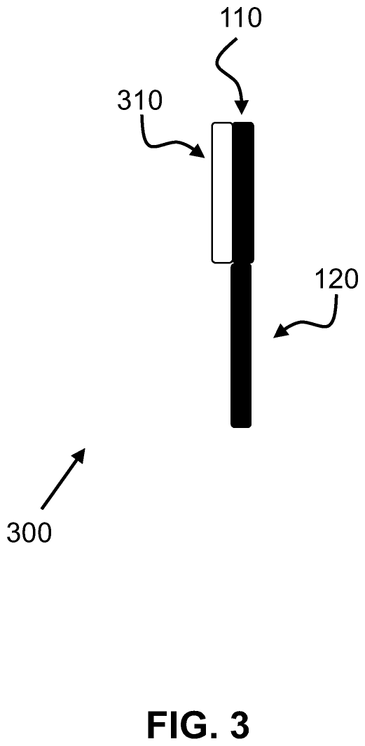

FIGS. 4-5 show according to an exemplary embodiment of the invention the porous membrane.

FIG. 6 shows according to an exemplary embodiment of the invention adhesives for attaching the membrane to the collector attachment section of the flexible substrate.

FIG. 7 shows according to an exemplary embodiment of the invention a side view of the wearable collector device adhered to a face mask.

FIG. 8 shows according to an exemplary embodiment of the invention a side view of the wearable collector device adhered to a face mask as well as the capability of the collector attachment section with the membrane to freely move with respect to the face mask as well the mask attachment section.

FIG. 9 shows according to an exemplary embodiment of the invention a view to the inner side of a pleated face mask (i.e. facing a person's mouth and/or nose) where the right view shows the pleats being expanded/stretched relative to the left view.

### DETAILED DESCRIPTION

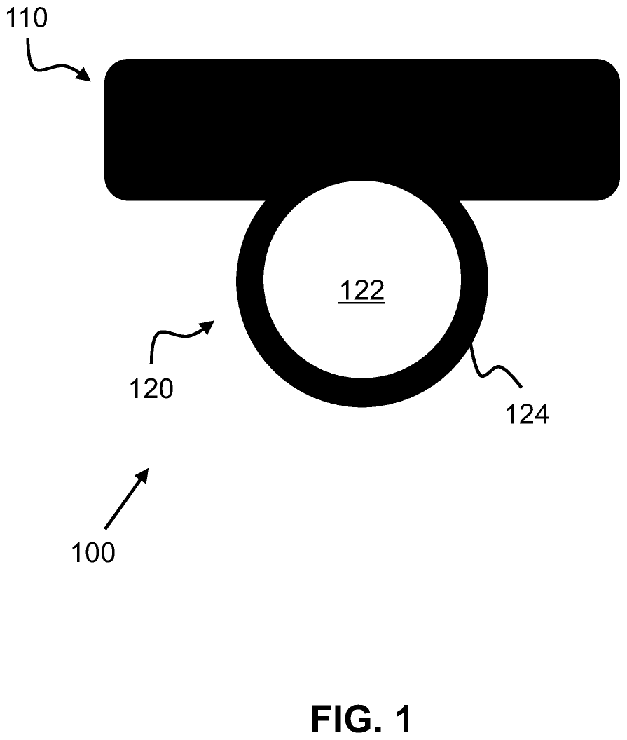

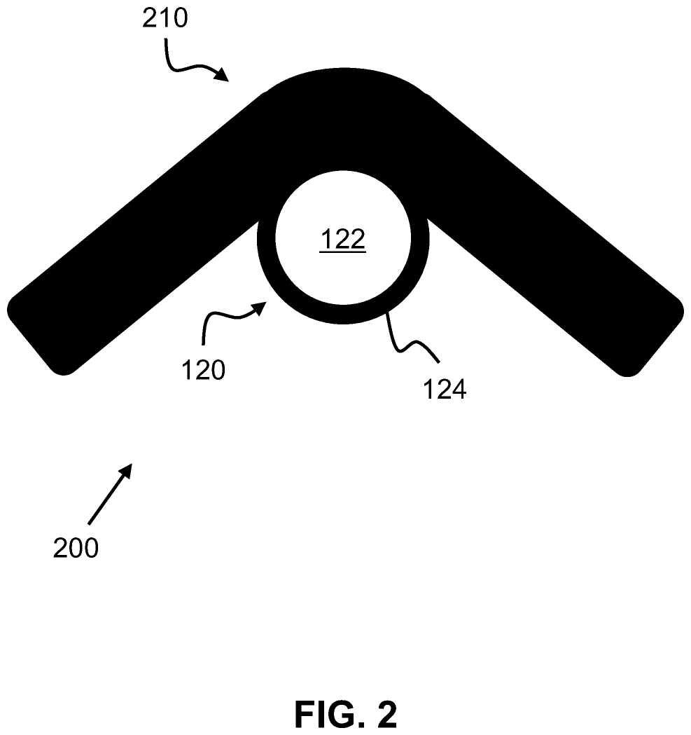

In one embodiment, the invention is a wearable collector device to collect biomarkers, pathogens, or viral loads through exposure from breath-derived droplets. The wearable collector device has a single piece flexible substrate 100 which distinguishes a mask attachment section 110 and a collector attachment section 120 situated below mask attachment section 110 (FIG. 1). In one example collector attachment section 120 is more or less centered with respect to mask attachment section 110 as depicted in FIG. 1. Collector attachment section 120 has an open area 122 more or less centered over collector attachment section 120. Attachment section 110 in FIG. 1 is shown as a rectangular shape, however, attachment section could vary in shape. For example, FIG. 2 shows substrate 200 with an angular shape or an L-shape for mask attachment section 210. Substrate 100 can be made of paper, plastic, or composite material as long as it is a flexible material. It is preferably a single piece which can be made via die-cutting, molding, hot embossing, or laser-cutting.

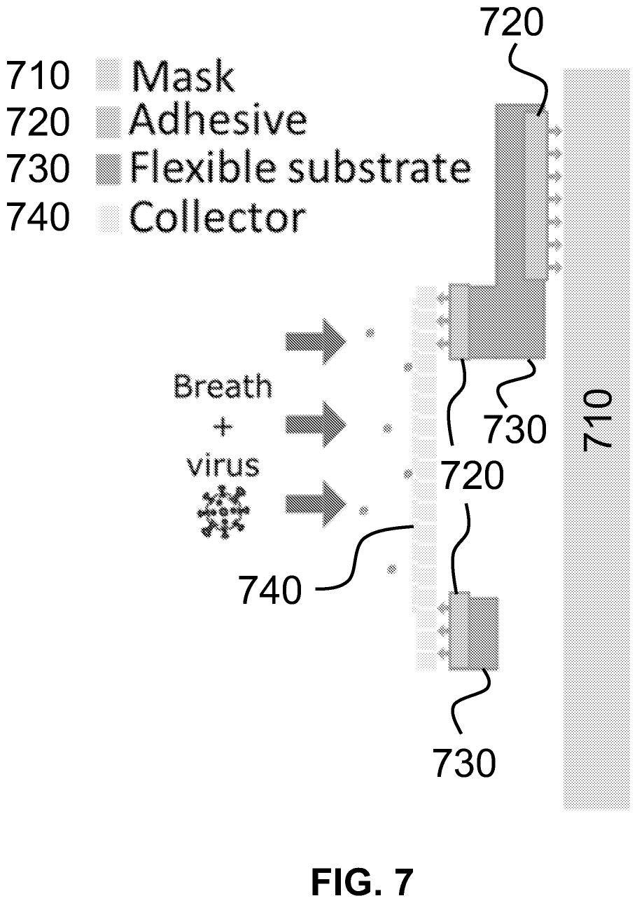

A double-sided adhesive 310 is sized to fit a side of mask attachment section 110. One side of adhesive 310 is suited to adhere to the side of mask attachment section 110, and the other side of adhesive 310 is suited to adhere to an inner surface of a mask as shown in FIGS. 7-9. In one embodiment, the mask is a pleated mask, and mask attachment section 110 is suited and capable of adhering to just a single pleat. This would make is easy for a person to stretch their mask without interfering with the position of the mask attachment section and therewith the wearable collector device as shown in FIGS. 7-9.

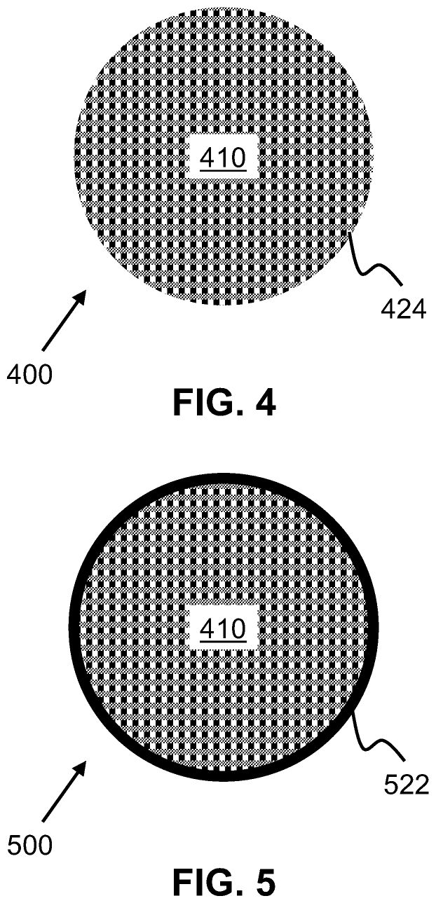

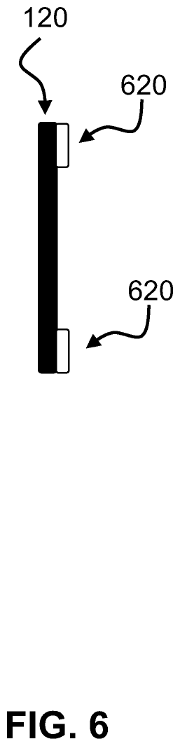

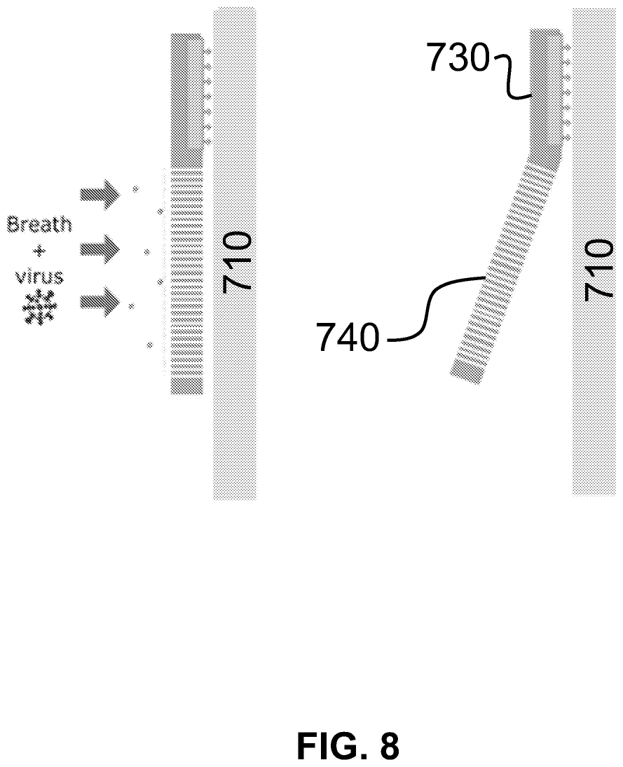

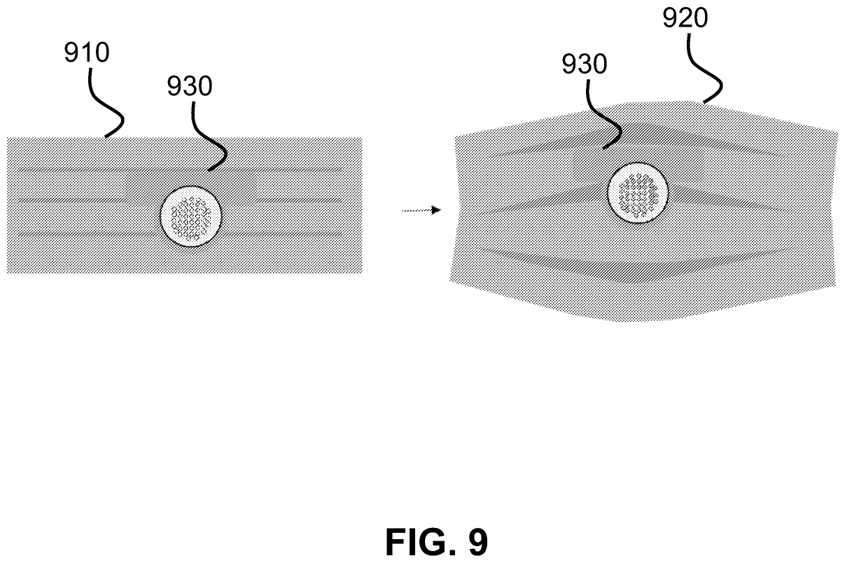

A membrane 400 is sized to cover at least the open area 122 of the collector attachment section 120. FIG. 4 shows membrane 400 where the entire membrane, including outer-edge area 424, is a porous membrane with the porous area indicated by 410. FIG. 5 shows membrane 500 distinguishing a porous area 510 and an outer-edge area 522 where outer-edge area 522 is not porous. In either case, outer edge areas 422, 522 are the areas that can be removably attached or adhere to collector attachment section 120 by means of for example adhesives 620 as shown in FIG. 6. It is key that porous membrane can be easily attached as well as removed from the wearable collector device. It is further key that collector attachment section 120 with membrane 400, 500 is capable of freely moving when the wearable collector device is attached to a mask such that movement from the mask or movement from the person wearing the wearable collector device does not interfere with the membrane (FIGS. 8-9). The membrane can be placed on either side of the collector attachment section 120.

The membrane can be made out of polycarbonate, nitrocellulose, aluminum oxide, or the like. The pore size of the membrane is in the range of 50 nm-5 μm and should be capable of collecting biomarkers, pathogens, or viral loads through exposure from breath-derived droplets.

The wearable collector device could be outfitted with sensor preferably in conjunction with the membrane to detect the biomarkers, pathogens, or viral loads. For instance, the collector could be integrated to with colorimetric, electrochemical, or optical sensors among others. The wearable collector device could further be outfitted with visual indicators to visualize detection of collected levels of the biomarkers, pathogens, or viral loads. For instance, the collector could be coupled with a lateral flow assay indicating the presence of a specific target.

The wearable collector device can be adhered to the inner surface of a face-covering as described above and is then ready to be used. A preferred time frame for usage is from minutes to hours range, with larger collection times providing a higher quantity of captured biomarkers. When ready the wearable patch as a whole or only membrane can be removed from the face covering and then membrane can be analyzed and e.g. be tested for SARS-CoV-2. Various tests can be performed, which are know in the art, such as e.g. polymerase chain reaction, loop-mediated isothermal amplification and immunoassays.

Additional teachings, embodiments and/or data can be found in U.S. Application No. 63/181,049 filed Apr. 28, 2021 to which this application claims the benefit for priority, and which is hereby incorporated by reference for all that it teaches.

What is claimed is:

1. A wearable collector device, comprising:
(a) a single piece flexible substrate, wherein the substrate distinguishes a mask attachment section and a collector attachment section situated below of the mask attachment section, wherein the collector attachment section has an open area more or less centered over the collector attachment section;
(b) a double-sided adhesive sized to fit a side of the mask attachment section, wherein one side of the adhesive is suited to adhere to the side of the mask attachment section, and wherein the other side of the adhesive is suited to adhere to an inner surface of a mask; and
(c) a porous membrane, wherein the membrane is sized to cover at least the open area of the collector attachment section, and wherein the membrane is sized to be removably attached to the collector attachment section either the same side of the mask attachment area or the other side of the mask attachment area, wherein the membrane has pores size in the range of 50 nm-5 μm and capable of collecting biomarkers, pathogens, or viral loads through exposure from breath-derived droplets, and wherein the membrane is capable freely moving with respect to the inner surface of the mask while the mask is worn by a person.

2. The wearable collector device as set forth in claim 1, wherein the substrate is made of paper, plastic, or composite material.

3. The wearable collector device as set forth in claim 1, wherein the open area is more or less centered with respect to the collector attachment section.

4. The wearable collector device as set forth in claim 1, wherein the collector attachment section has a rectangular shape or an angled shape.

5. The wearable collector device as set forth in claim 1, wherein the mask has pleats and wherein the other side of the adhesive is suited to adhere to a single pleat.

6. The wearable collector device as set forth in claim 1, further comprises sensors to detect collected levels of the biomarkers, pathogens, or viral loads.

7. The wearable collector device as set forth in claim 1, further comprises visual indicators to visualize detection of collected levels of the biomarkers, pathogens, or viral loads.

8. The wearable collector device as set forth in claim 1, wherein the membrane is made out of polycarbonate, nitrocellulose or aluminum oxide.

9. A method of collecting biomarkers, pathogens, or viral loads through exposure from breath-derived droplets, comprising:
(a) adhering a wearable collector device to an inner surface of a face mask, wherein the wearable collector device comprises:
(i) a single piece flexible substrate, wherein the substrate distinguishes a mask attachment section and a collector attachment section situated below the mask attachment section, wherein the collector attachment section has an open area more or less centered over the collector attachment section;
(ii) a double-sided adhesive sized to fit a side of the mask attachment section, wherein one side of the adhesive is suited to adhere to the side of the mask attachment section, and wherein the other side of the adhesive is suited to adhere to the inner surface of the face mask; and
(iii) a porous membrane, wherein the membrane is sized to cover at least the open area of the collector attachment section, and wherein the membrane is sized to be removably attached to the collector attachment section either the same side of the mask attachment area or the other side of the mask attachment area, wherein the membrane has pores size in the range of 50 nm-5 μm and capable of collecting the biomarkers, pathogens, or viral loads through exposure from breath-derived droplets, and wherein the membrane is capable freely moving with respect to the inner surface of the mask while the mask is worn by a person; and
(b) removing the membrane from the wearable collector device for analysis of collected biomarkers, pathogens, or viral loads.

10. The method as set forth in claim 1, wherein the substrate is made of paper, plastic, or composite material.

11. The method as set forth in claim 1, wherein the open area is more or less centered with respect to the collector attachment section.

12. The method as set forth in claim 1, wherein the collector attachment section has a rectangular shape or an angled shape.

13. The method as set forth in claim 1, wherein the mask has pleats and wherein the other side of the adhesive is suited to adhere to a single pleat.

14. The method as set forth in claim 1, further comprises sensors to detect collected levels of biomarkers, pathogens, or viral loads.

15. The method as set forth in claim 1, further comprises sensing collected levels of biomarkers, pathogens, or viral loads.

16. The method as set forth in claim 1, further comprises visual indicators to visualize detection of collected levels of the biomarkers, pathogens, or viral loads.

17. The method as set forth in claim 1, further comprises visualizing collected levels of the biomarkers, pathogens, or viral loads.

18. The method as set forth in claim 1, wherein the membrane is made out of polycarbonate, nitrocellulose or aluminum oxide.

19. The method as set forth in claim 1, wherein the substrate is made from die-cutting, molding, hot embossing, or laser-cutting.
